(19) United States
(12) Patent Application Publication
(10) Pub. No.: US 2026/0259145 A1
(43) Pub. Date: Sep. 3, 2026

(54) MULTI-LINED DETECTION DEVICE

(71) Applicant: LEADWAY (HK) LIMITED, Hong Kong (CN)

(72) Inventors: Chao CUI, Hangzhou (CN); Yun LING, Hangzhou (CN); Tianhao LI, Hangzhou (CN); Chunming WANG, Hangzhou (CN)

(73) Assignee: LEADWAY (HK) LIMITED, Hong Kong (CN)

(21) Appl. No.: 18/846,661

(22) PCT Filed: Nov. 18, 2022
(86) PCT No.: PCT/CN2022/132931

§ 371 Date: Sep. 13, 2024

(30) Foreign Application Priority Data
Apr. 8, 2022 (CN) 202210370196.8
Apr. 26, 2022 (CN) 202210447619.1

(51) Int. Cl. G01N 21/78; B01L 3/00; G01N 21/77

(52) U.S. Cl. CPC G01N 21/78; B01L 3/5023; B01L 2200/0689; B01L 2300/0816; B01L 2300/0825; B01L 2300/12; G01N 2021/7759

(57) ABSTRACT

The present invention relates to a multi-lined detection device, including a bottom card, at least two test strips located above the bottom card and arranged side by side, and at least two sealing films located above the bottom card and the test strips. Wherein, the at least two sealing films each form a cavity with the bottom card in the area where the test strip is located, and a bonding band connecting said sealing film to the bottom card is formed around the cavity. The test strip is received in the cavity. The bottom card forms an incompletely disconnected cutting line in an area between two adjacent bonding bands, and two adjacent sealing films are completely disconnected in the area between the two adjacent bonding bands.

### FIELD OF TECHNOLOGY

The present invention relates to a detection device, in particular to a multi-lined detection device suitable for automated production.

### TECHNICAL BACKGROUND

Disposable detection kits for detection of diseases or other physical conditions using body fluids such as urine, blood or other tissue fluids of a human body have been commonly used all over the world, and their applications can be in laboratories operated by professionals, or at home, schools, shopping malls, road checkpoints, customs, etc. operated by non-professionals themselves. As shown in FIG. 1 and FIG. 2, such a detection kit 1 generally includes an upper cover 2, a bottom plate 3 and a test strip 4, wherein the upper cover 2 and the bottom plate 3 are combined in such a manner as buckling, welding, glue adhering or the like, and the test strip 4 is arranged on the bottom plate 3 and located between the upper cover 2 and the bottom plate 3. The upper cover 2 includes a sample addition hole 5 and an observation hole 6. A liquid sample for detection is applied to a test strip via the sample addition hole 5, and after the end of the detection, a detection result on the test strip is observed via the observation hole 6, and determined accordingly.

The upper cover 2 and the bottom plate 3 of such detection kit as shown in FIG. 1 and FIG. 2, are made of plastic articles that are thermally injection molded, and they account for a large portion of the cost of the whole detection kit. Both the upper cover 2 and the bottom plate 3 have a certain height, and after their combination, the size of the whole detection kit 1 increases, and is much larger than the size of the test strip 4, increasing storage and transportation costs. Moreover, due to long degradation time of plastics, usage of a large amount of plastics does not comply with the concept of environmental protection. Such detection kit 1 has been packaged in an aluminum foil bag in factory to avoid bumping against the test strips in the detection kit 1 during transportation; and an operator has to tear the aluminum foil bag when the kit is to be used, and then takes out of the detection kit 1 for detection, so that the operation steps are tedious. Furthermore, such detection detection kit is not suitable for manufacturing by automated equipment, but requires a large amount of human power for manual assembly, the assembly process is boring and cumbersome, and long time of operation is prone to resulting in fatigue, burnout and errors, reducing the qualification rate of products, and increasing labor costs.

As the disposable detection kits have been widely used, in order to reduce the purchase costs of users, it is necessary to meet these needs with the lowest possible product cost and convenient usage mode. Particularly in the case of spreading of epidemic diseases, how to quickly promote such detection kits so that more people can afford to use and are willing to use such rapid detection kit products is a problem that needs to be solved at present.

An objective of the present invention is to provide a multi-lined detection device, by which automated production is easy to realize. The device is small in size, is low in production, packaging and transportation costs, and uses little plastic materials, so that it is very environmentally friendly. When the multi-lined detection device is split into at least two single detection devices, the sealed packaging of adjacent detection devices is not affected.

### SUMMARY OF THE INVENTION

In order to solve the above technical problems, the present invention provides a multi-lined detection device, comprising a bottom card, at least two test strips located above the bottom card and arranged side by side, and at least two sealing films located above the bottom card and the test strips, the thickness of the bottom card is greater than the thickness of the sealing film. Wherein, the at least two sealing films each form a cavity with the bottom card in the area where the test strip is located, and a bonding band connecting said sealing film to the bottom card is formed around the cavity. The test strip is received in the cavity. The bottom card forms a cutting line, which is incompletely disconnected, in an area between two adjacent bonding bands. The two adjacent sealing films are completely disconnected in the area between the two adjacent bonding bands.

Further improvement of the present invention lies in that the incompletely disconnected cutting line on the bottom card comprises a discontinuous cutting line, a continuous cutting line or a combination thereof; the discontinuous cutting line is formed by connecting segments spaced in sequence and fractures penetrating through the bottom card; and the continuous cutting line is a cutting line not penetrating through the bottom card.

Further improvement of the present invention lies in that the length of the connecting segment of the discontinuous cutting line ranges from 0.1 mm to 3.0 mm, the length of the fracture of the discontinuous cutting line ranges from 0.5 mm to 10.0 mm, and the cutting depth of the continuous cutting line ranges from one fifth to four fifths of the thickness of the bottom card.

Further improvement of the present invention lies in that the bottom card has a first end and a second end in a direction parallel to the cutting line, wherein the shortest distance from the first end to the bonding band is greater than the shortest distance from the second end to the bonding band, and the bottom card forms a completely disconnected opening, which is “U” shaped or “V” shaped, at the junction of the first end thereof and the cutting line.

Further improvement of the present invention lies in that the bonding band bonding the sealing film to the bottom card is closed, and the cavity for receiving the test strip is sealed by the closed bonding band.

Further improvement of the present invention lies in that the closed bonding band comprises an intermediate portion and two ends connected to the two ends of the intermediate portion, respectively, wherein the width of at least the end close to the first end of the bottom card gradually decreases in the direction from the intermediate portion of the bonding band to the two ends of the bonding band.

Further improvement of the present invention lies in that the intermediate portion is two straight lines parallel to each other, and at least the end close to the first end of the bottom card is of a circular structure or a triangular structure without a bottom edge or a trapezoidal structure without a bottom edge.

Further improvement of the present invention lies in that a completely disconnected notch is formed between two adjacent sealing films.

Further improvement of the present invention lies in that the test strip is bonded to the bottom card through viscose.

Further improvement of the present invention lies in that the bottom card is made of a PE or PP material with the thickness ranging from 0.1 mm to 0.5 mm; and the sealing film is made of a PE or PP or PET material with the thickness ranging from 0.05 mm to 0.20 mm.

### Beneficial Effects

The present is small in size and uses a little amount of materials, so that the packaging and transportation costs are low. Furthermore, since a relatively flexible material that can be rolled is used, it is suitable for automated production, greatly reducing labor intensity and production costs. In addition, since the two adjacent sealing films are completely disconnected, when the bottom card is torn, the adjacent sealing films are not torn and the sealed packaging of the adjacent detection devices is not affected.

Moreover, in the technical solution of the present invention that the sealing film is completely cut off, when two adjacent detection devices are torn, the tearing force cannot be transferred to the sealing films of the adjacent detection devices because the two adjacent sealing films are completely disconnected and have no connection between each other and generate no implication between them, thus not affecting the bonding formed by the adjacent sealing films and the bottom card, so that the sealed packaging of the adjacent detection devices will not be damaged.

Further, the present invention forms a completely disconnected notch between the two adjacent sealing films, and the completely disconnected notch provides convenience for detection of whether the two adjacent sealing films are completely disconnected in the area between the two adjacent bonding bands, as the formation of the completely disconnected notch is more convenient for visual observation. This design improves the probability of complete disconnection between two adjacent sealing films and reduces the probability of affecting adjacent sealing films when a single detection device is torn, resulting in better beneficial effects.

### DESCRIPTION OF THE DRAWINGS

FIG. 1 is a stereoscopic exploded view of a detection kit in the prior art;

FIG. 2 is a stereoscopic schematic view of FIG. 1 after combination;

FIG. 3 is an exploded view of a first embodiment of a multi-lined detection device of the present invention;

FIG. 4 is a partially combined view of FIG. 3;

FIG. 5 is a top view of the multi-lined detection device of the present invention after removal of sealing films;

FIG. 6 is a top view of the sealing film of the multi-lined detection device of the present invention;

FIG. 7 is a top view of FIG. 3 after combination (the parts obscured by the sealing films are shown by dashed lines);

FIG. 8 is a partially enlarged schematic view within circle Ain FIG. 5;

FIG. 9 is a partially enlarged schematic view within circle B in FIG. 6;

FIG. 10 is a partially enlarged schematic view within circle C in FIG. 7;

FIG. 11 is a sectional view of FIG. 7 along line A-A;

FIG. 12 is a partially enlarged view within circle Din FIG. 11;

FIG. 13 is schematic view of a modified design of FIG. 12;

FIG. 14 is a schematic view of another modified design of FIG. 12;

FIG. 15 is a schematic view of another modified design of FIG. 12;

FIG. 16 is schematic view of a modified design of FIG. 5;

FIG. 17 is a partially enlarged schematic view of a part within circle E in FIG. 16 along line B-B;

FIG. 18 is a schematic view of a modified design of FIG. 8;

FIG. 19 is a schematic view of another modified design of FIG. 8;

FIG. 20 is a top view of a single detection device of after removal of sealing films, in which the shadow lines represent a bonding band;

FIG. 21 is schematic view of a modified design of FIG. 20;

FIG. 22 is a schematic view of another modified design of FIG. 20;

FIG. 23 is a schematic view of another modified design of FIG. 20;

FIG. 24 is a partially enlarged schematic view within circle F in FIG. 20;

FIG. 25 is a partially enlarged schematic view within circle G in FIG. 21;

FIG. 26 is a partially enlarged schematic view within circle H in FIG. 22;

FIG. 27 is a partially enlarged schematic view within circle I in FIG. 23;

FIG. 28 is a schematic view of a modified design of FIG. 3; and

FIG. 29 is a schematic top view after one of the detection devices in the multi-lined detection device of the present invention is torn off.

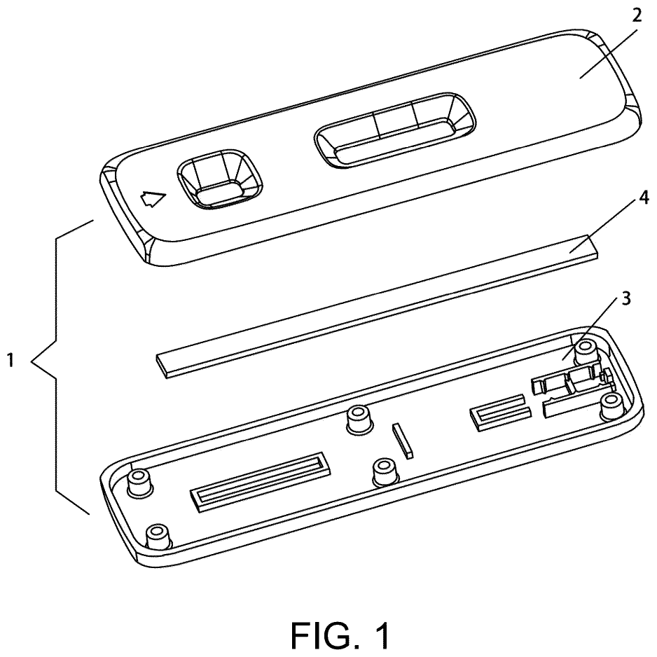

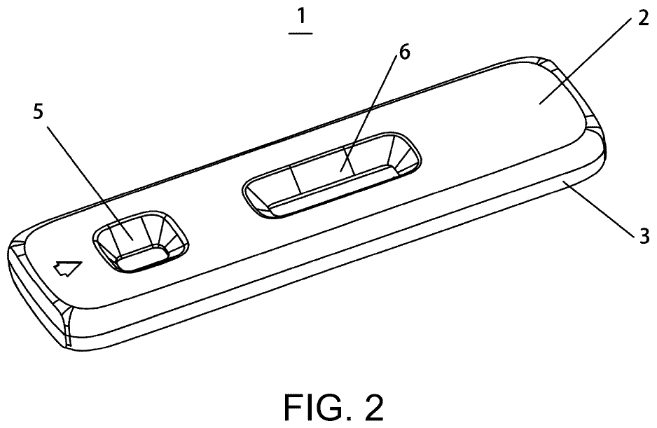

### DETAILED DESCRIPTION OF THE EMBODIMENTS

The technical solutions of the present invention will be further specified below through embodiments in conjunction with the drawings.

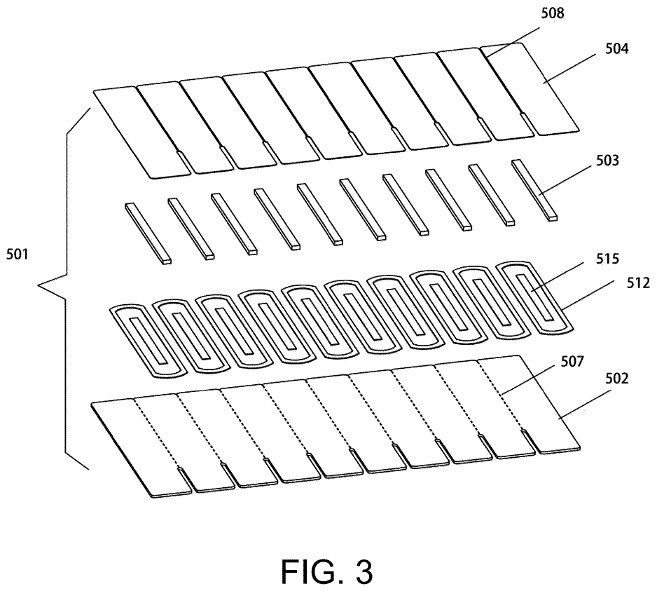

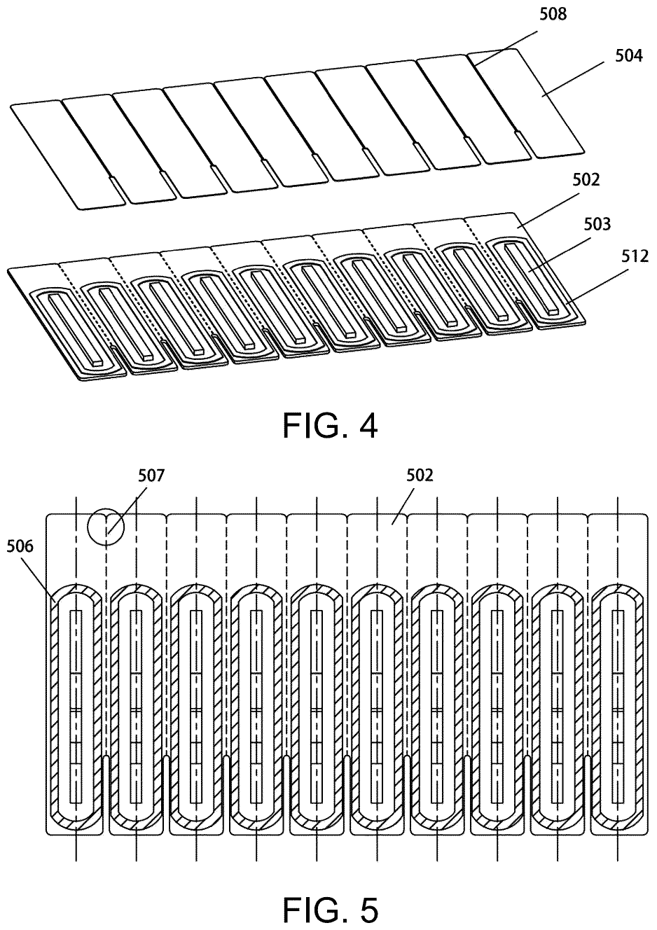

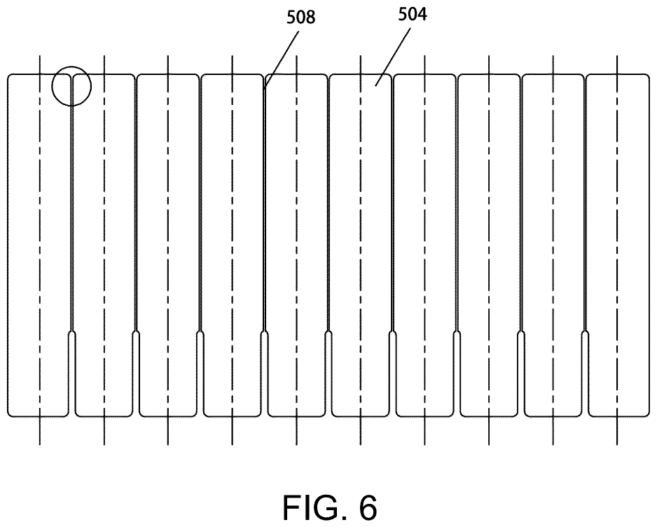

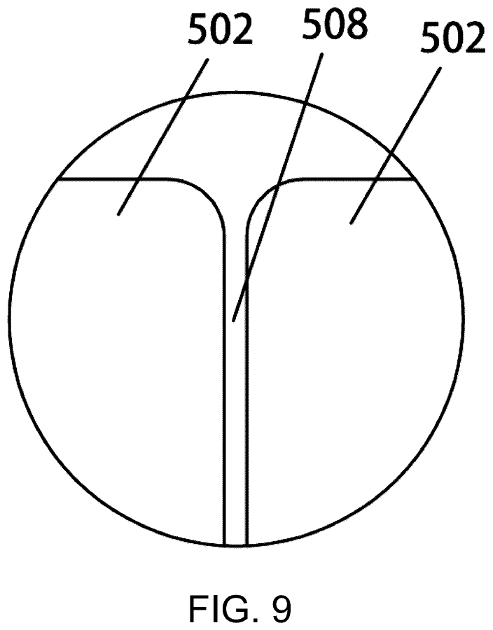

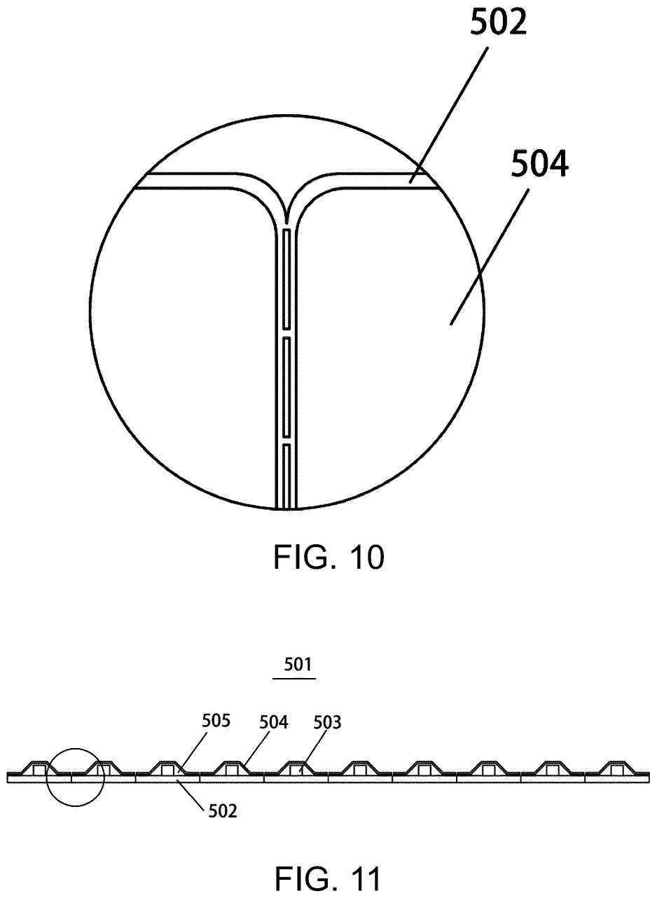

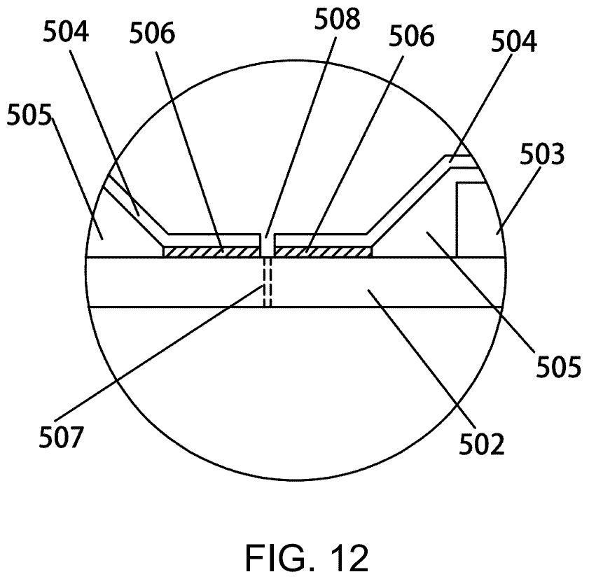

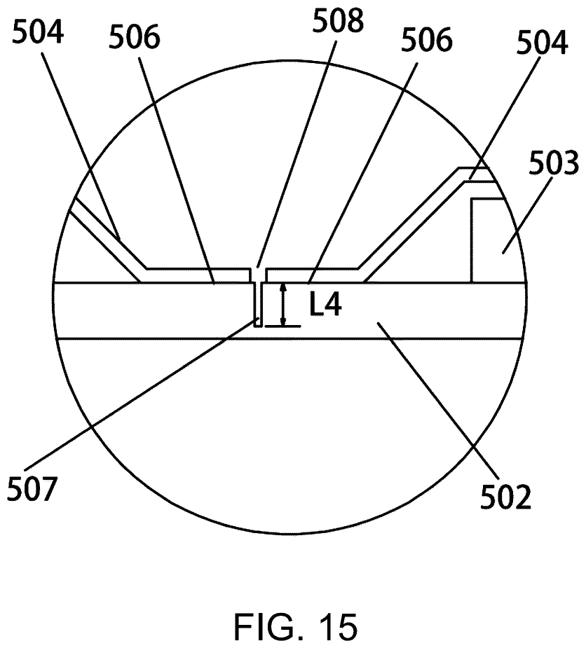

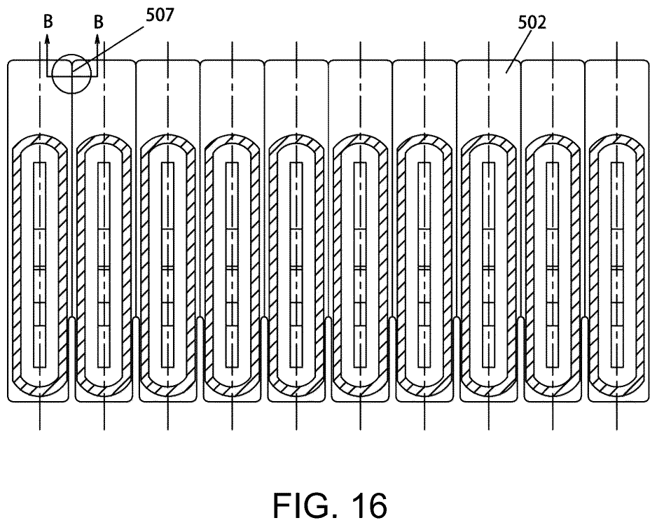

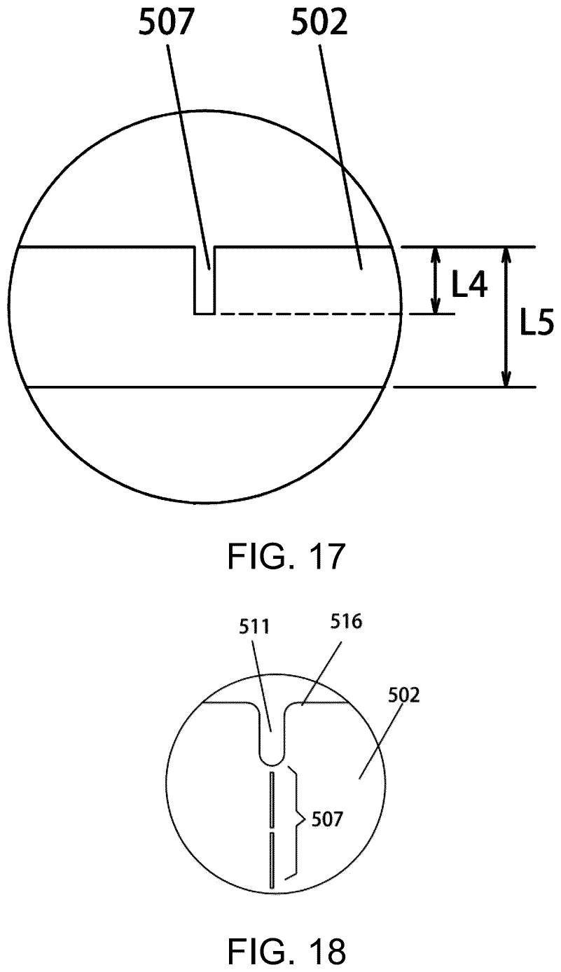

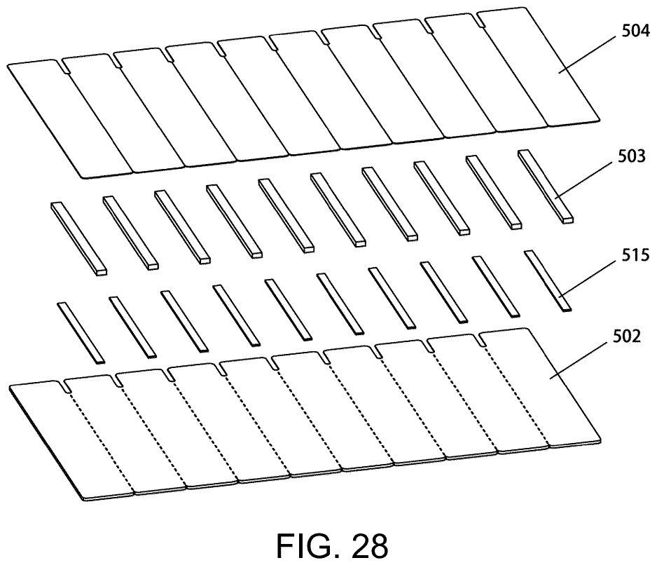

Please refer to FIGS. 3 to 29, the present invention relates to a multi-lined detection device 501, including a bottom card 502, at least two test strips 503 located above the bottom card and arranged side by side, and at least two sealing films 504 located above the bottom card and the test strips. Wherein, these sealing films 504 each form a cavity 505 with the bottom card 502 in the area where the test strip 503 is located, and a bonding band 506 connecting said sealing film to the bottom card is formed around the cavity (please refer to FIGS. 12 to 15). The test strip 503 is received in the cavity 505. The bottom card 502 forms an incompletely disconnected cutting line 507 (as shown by the dashed line in FIG. 5) in an area between two adjacent bonding bands 506. In the embodiments shown in FIGS. 12 to 15, two adjacent sealing films 504 are completely disconnected in the area between the two adjacent bonding bands 506 to form a completely disconnected notch 508. The notch 508 causes the two sealing films 504 to form a gap in the area between the two adjacent bonding bands 506, as shown in FIG. 9. The gap may be between 0.05 mm to 2.0 mm, preferably, 0.1 mm to 0.5 mm. The design has the advantages that when two adjacent detection devices are torn, the tearing force cannot be transferred to the sealing films 504 of the adjacent detection devices because the two adjacent sealing films 504 are completely disconnected and have no connection between each other and generate no implication between them, thus not affecting the bonding formed by the adjacent sealing films 504 and the bottom card 502, so that the sealed packaging of the adjacent detection devices will not be damaged. Further, the completely disconnected notch 508 provides convenience for detection of whether the two adjacent sealing films 504 are completely disconnected in the area between the two adjacent bonding bands 506, as the formation of the completely disconnected notch 508 is more convenient for detection through visual observation (not only including detection through human visual observation but also including detection through automated visual observation of equipment). This design improves the reliability of completely disconnecting two adjacent sealing films 504 and reduces the probability of affecting adjacent sealing films 504 when a single detection device is torn, resulting in better beneficial effects. The test strip 503 is bonded to the bottom card 502 through viscose 515.

The bottom card 502 is preferably made of a moisture-proof and oxidation-resistant PE or PP material, with its thickness preferably ranging from 0.1 mm to 0.5 mm, more preferably ranging from 0.2 mm to 0.35 mm. The sealing film 504 is preferably made of a moisture-proof and oxidation-resistant PE or PP or PET material, with its thickness preferably ranging from 0.05 mm to 0.20 mm, more preferably ranging from 0.08 mm to 0.15 mm. Since the thickness of the bottom card 502 is much greater than the thickness of the sealing film 504, the bottom card 502 has moderate rigidity and higher strength, while the sealing film 504 is softer and has better stretching resistance and tearing resistance performances, and both of them are suitable for producing products of the present invention on automated equipment.

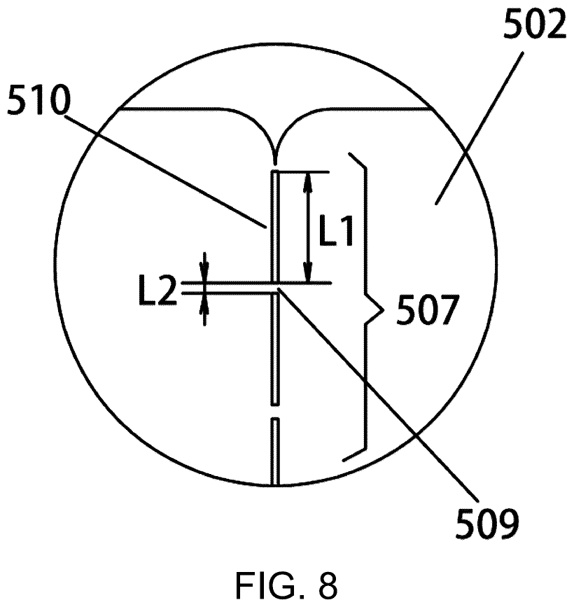

In the embodiment shown in FIG. 8, the incompletely disconnected cutting line 507 on the bottom card 502 is a discontinuous cutting line, which is formed by connecting segments 509 spaced in sequence and fractures 510 penetrating through the bottom card. The longer the connecting segment 509 is, the better the connecting stability is (the lower the probability of improper separation of the bottom card 502 is), but the larger the difficulty of separating the bottom card 502 along the incompletely disconnected cutting line 507 is; and in contrary, the shorter the connecting segment 509 is, the worse the connecting stability is (the higher the probability of improper separation of the bottom card 502 is), but the smaller the difficulty of separating the bottom card 502 along the incompletely disconnected cutting line 507 is. Therefore, it is necessary to properly select the lengths of the connecting segment 509 and the fracture 510. Preferably, the length L1 of the fracture 510 of the discontinuous cutting line ranges from 0.5 mm to 10.0 mm (ranging from value A to value B means greater than or equal to value A and less than or equal to value B, the same in the full text), and the length L2 of the connecting segment 509 of the discontinuous cutting line ranges from 0.1 mm to 3.0 mm; and more preferably, the length L1 of the fracture 510 of the discontinuous cutting line ranges from 1.0 mm to 5.0 mm, and the length L2 of the connecting segment 509 of the discontinuous cutting line ranges from 0.5 mm to 1.5 mm.

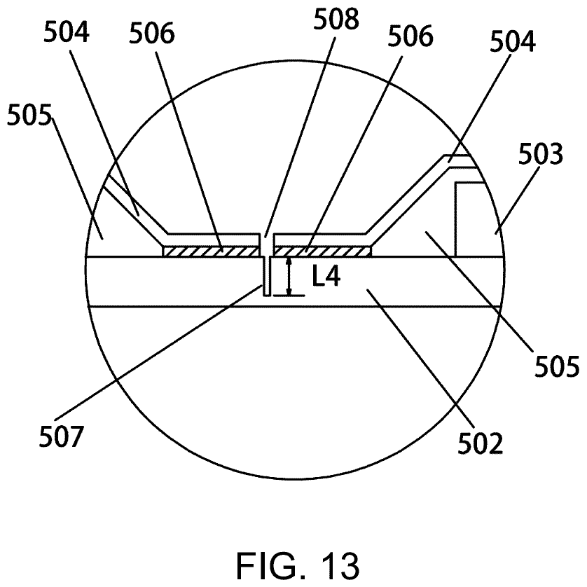

The side of the bottom card 502 facing the sealing film 504 is an upper surface, the back side of which is a lower surface. In the embodiments shown in FIGS. 13, 15 and 17, the incompletely disconnected cutting line 507 on the bottom card 502 is a continuous cutting line not penetrating through the upper and lower surfaces of the bottom card. Preferably, the cutting depth L4 of this continuous cutting line ranges from one fifth to four fifths of the thickness L5 of the bottom card. The continuous cutting line is preferably located on the upper surface of the bottom card, but may also be located on the lower surface of the bottom card.

In another embodiment (not shown in the figure), the incompletely disconnected cutting line 507 on the bottom card 502 may also be a combination of the discontinuous cutting line and the continuous cutting line as previously described, i.e., one part of the incompletely disconnected cutting line 507 is the discontinuous cutting line as described in FIGS. 8, 12 and 14 and the other part is the continuous cutting line as shown in FIGS. 13, 15 and 17.

In the embodiments shown in FIGS. 12 to 15, the at least two adjacent sealing films 504 are bonded to the bottom card 502 at the edge of its area between the two adjacent bonding bands 506. This embodiment has the advantages that the multi-lined detection device 501 in the present invention is compact in structure and small in size.

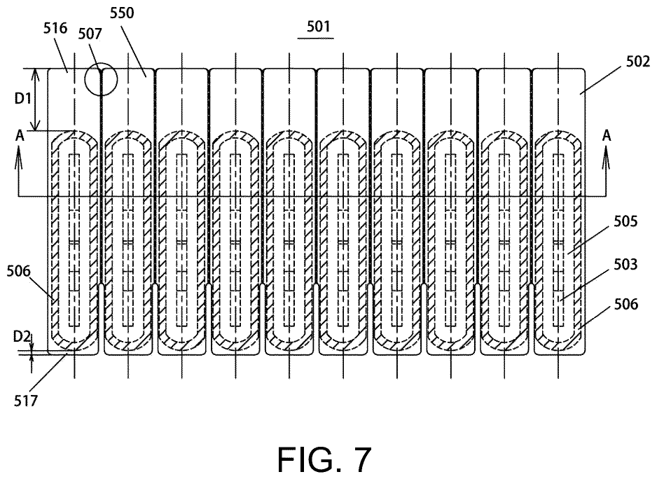

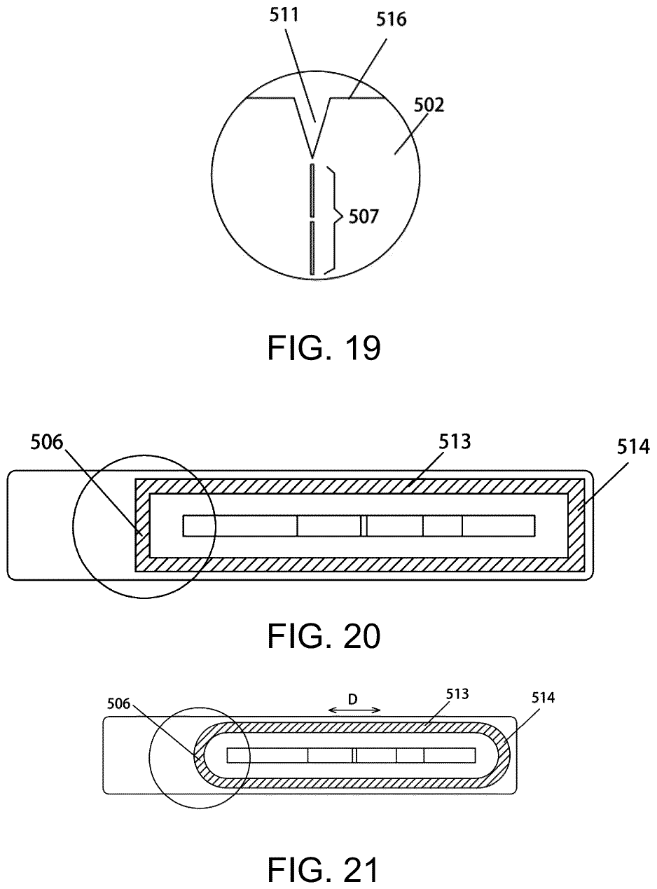

Please refer to FIG. 7, the bottom card 502 has a first end 516 and a second end 517 in a direction parallel to the incompletely disconnected cutting line 507, wherein the shortest distance “D1” from the first end 516 to the bonding band 506 is greater than the shortest distance “D2” from the second end 517 to the bonding band 506. As shown in FIGS. 18 and 19, the bottom card 502 forms a completely disconnected opening 511, which is “U” shaped (as shown in FIG. 18) or “V” shaped (as shown in FIG. 19), at the junction of the first end 516 thereof and the incompletely disconnected cutting line 507. The purpose of providing the completely disconnected opening 511 is to use the completely disconnected opening 511 as a starting point for tearing, thereby facilitating separation of the two adjacent single detection devices 550. The first end 516 typically serves as a holding end for an operator to tear off the sealing film 504 from the bottom card 502. Preferably, the completely disconnected opening 511 is located at the first end 516 of the bottom card 502, thus after tearing off the detection device from the completely disconnected opening 511, the sealing film 504 can be torn from the bottom card 502 directly without changing hands to expose the test strip 503 for detection, instead of changing hands and subsequently turning over the torn single detection device 550 (inverted) to tear the sealing film 504 from the bottom card 502, so that the operation is more convenient.

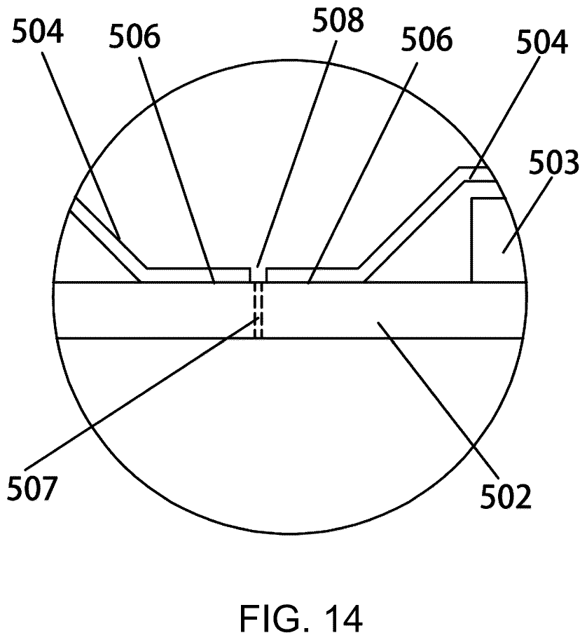

Please refer to FIGS. 7, 11 and FIGS. 20 to 23, the bonding band 506 that bonds the sealing film 504 to the bottom card 502 is a closed ring, and the cavity 505 receiving the test strip 503 is sealed by the closed bonding band 506. The closed ring described in the present invention means that the bonding band 506 is closed, rather than limiting its shape to a circular ring; and on the contrary, the structure may be as shown in FIGS. 20 to 23 or may be a structure developed according to FIGS. 20 to 23. In one embodiment, the bonding band 506 is formed by a binder 512 (e.g., glue or double-sided adhesive tape, etc.) (as shown in FIG. 3). In another embodiment, no binder is used and the bonding band 506 is formed by fusing the bottom card 502 and the sealing film 504 under pressure and heat (as shown in FIGS. 14, 15 and 28). In another embodiment, the bottom card 502 has a thermally adhesive film on its side facing the sealing film 504, the thermally adhesive film bonding the sealing film 504 to the bottom card 502 when being subjected to pressure and heat, so as to form said bonding band 506 (not shown in the figure).

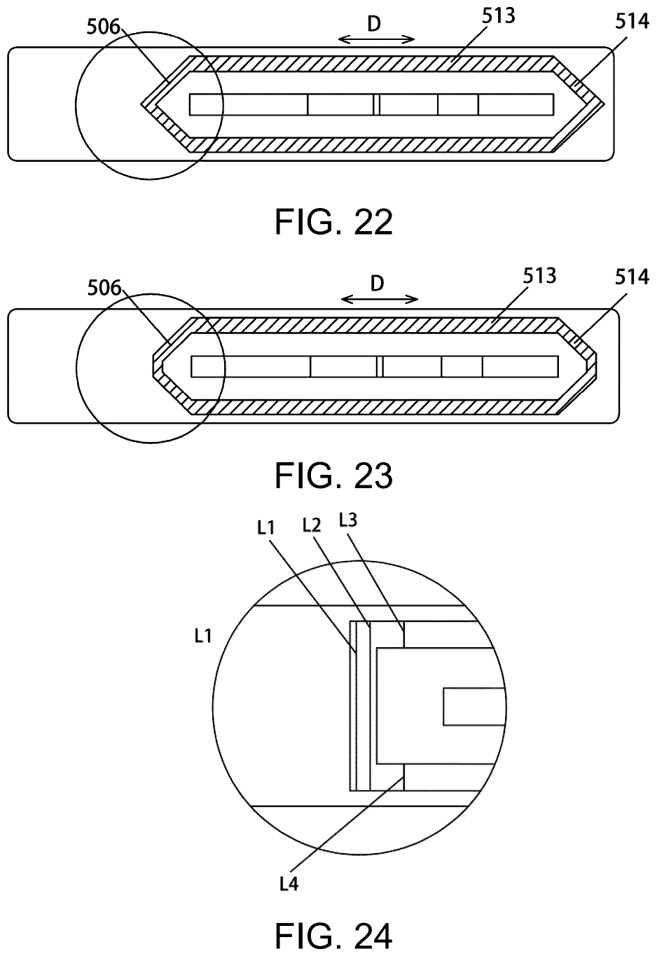

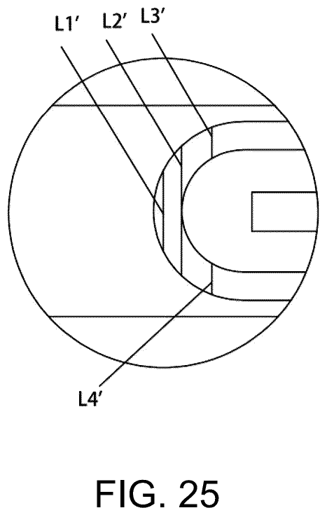

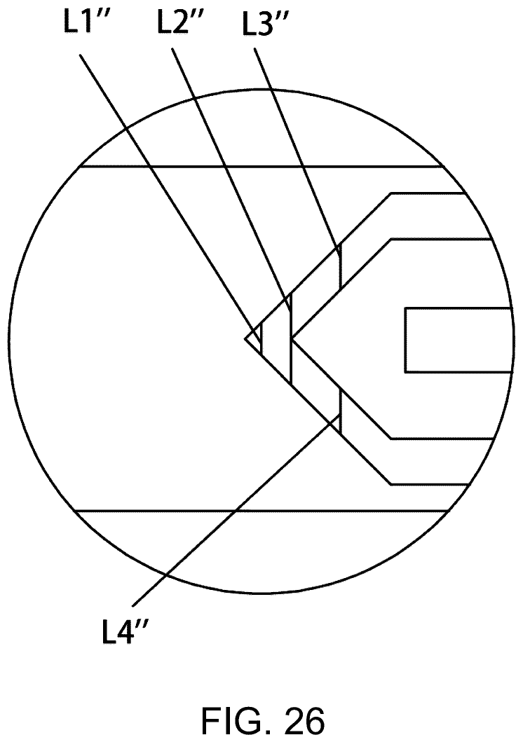

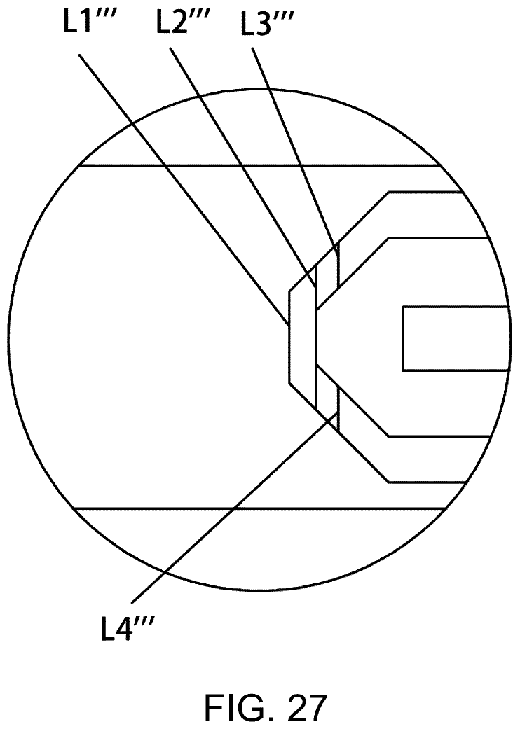

Please refer to FIGS. 20 to 27, the closed bonding band 506 includes an intermediate portion 513 and two ends 514 connected to the two ends of the intermediate portion, respectively. The intermediate portion 513 preferably is two straight lines parallel to each other. In the embodiments shown in FIG. 20 and FIG. 24, the width of the end 514 is substantially constant. In the embodiment shown in FIGS. 21 to 23 and FIGS. 25 to 27, the width of the end 514 gradually decreases from the intermediate portion of the bonding band to the two ends of the bonding band (in the direction shown by arrow “D” in FIGS. 21 to 23). Preferably, the end 514 is of a circular structure (as shown in FIGS. 21 and 25) or a triangular structure without a bottom edge (as shown in FIGS. 22 and 26) or a trapezoidal structure without a bottom edge (as shown in FIGS. 23 and 27). In another embodiment, the two ends 514 may also be a combination of any of the structures as shown in FIGS. 21 to 23. In contrast to the embodiment shown in FIG. 24, the embodiments shown in FIGS. 25 to 27 have the advantages that the lengths of tear lines L1′, L2′, (L3′+L4′), tear lines L1″, L2″′, (L3″+L4″) and tear lines L1″′, L2″′, (L3″+L4″) are smaller than the lengths of tear lines L1, L2, and (L3+L4) in FIG. 24, respectively, i.e., L1′<L1, L2′<L2, (L3′+L4′)<(L3+L4), and so on for others. This has the advantages that in the tearing process, the bonding force between the sealing film 504 and the bottom card 502 is smaller, so that it is easier to tear and separate the sealing film 504 from the bottom card 502.

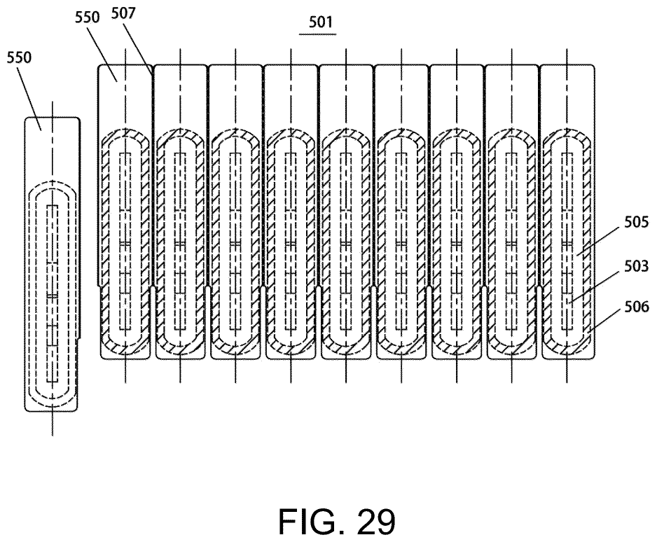

FIG. 29 is a schematic view of tearing off a single detection device 550 from the multi-lined detection device 501 along the incompletely disconnected cutting line 507.

The above description is only the specific implementations of the present invention, and is not thereby limiting the scope claimed for protection by the present invention. Any equivalent structures or equivalent process transformations made by using the description and the drawings of the present invention, directly or indirectly applied to other related technical fields, should be encompassed in the protection scope of the present invention.

1. A multi-lined detection device, comprising:
a bottom card, at least two test strips located above the bottom card and arranged side by side, and at least two sealing films located above the bottom card and the test strips, a thickness of the bottom card is greater than a thickness of the sealing film, the at least two sealing films each form a cavity with the bottom card in an area where the test strip is located, and a bonding band connecting the sealing film to the bottom card is formed around the cavity, the test strip is received in the cavity, and the bottom card forms a cutting line, which is incompletely disconnected, in an area between two adjacent bonding bands, and two adjacent sealing films are completely disconnected in the area between the two adjacent bonding bands.

2. The multi-lined detection device of claim 1, wherein the incompletely disconnected cutting line on the bottom card comprises a discontinuous cutting line, a continuous cutting line or a combination thereof; the discontinuous cutting line is formed by connecting segments spaced in sequence and fractures penetrating through the bottom card; and the continuous cutting line is a cutting line not penetrating through the bottom card.

3. The multi-lined detection device of claim 2, wherein a length of the connecting segment of the discontinuous cutting line ranges from 0.1 mm to 3.0 mm, a length of the fracture of the discontinuous cutting line ranges from 0.5 mm to 10.0 mm, and a cutting depth of the continuous cutting line ranges from one fifth to four fifths of the thickness of the bottom card.

4. The multi-lined detection device of claim 2, wherein the bottom card has a first end and a second end in a direction parallel to the cutting line, wherein a shortest distance from the first end to the bonding band is greater than a shortest distance from the second end to the bonding band, and the bottom card forms a completely disconnected opening, which is “U” shaped or “V” shaped, at the junction of the first end thereof and the cutting line.

5. The multi-lined detection device of claim 4, wherein the bonding band bonding the sealing film to the bottom card is closed, and the cavity for receiving the test strip is sealed by the closed bonding band.

6. The multi-lined detection device of claim 5, wherein the closed bonding band comprises an intermediate portion and two ends connected to two ends of the intermediate portion, respectively, wherein, a width of at least the end close to the first end of the bottom card gradually decreases in a direction from the intermediate portion of the bonding band to the two ends of the bonding band.

7. The multi-lined detection device of claim 6, wherein the intermediate portion is two straight lines parallel to each other, and at least the end close to the first end of the bottom card is of a circular structure or a triangular structure without a bottom edge or a trapezoidal structure without a bottom edge.

8. The multi-lined detection device of claim 1, wherein a completely disconnected notch is formed between the two adjacent sealing films.

9. The multi-lined detection device of claim 1, wherein the test strip is bonded to the bottom card through viscose.

10. The multi-lined detection device of claim 1, wherein the bottom card is made of a PE or PP material with a thickness ranging from 0.1 mm to 0.5 mm; and the sealing film is made of a PE or PP or PET material with a thickness ranging from 0.05 mm to 0.20 mm.
